SEQUENCE LISTING (2 sequences)

SEQ ID NO: 1 - protein, 107 aa, human
DIQMTQSPSSLSASVGDRVTITCRASQDISNFLNWYQQKPGKAPKLLIYYTSTLHSGVPSRFSGSGSGTDFTLTISSLQPEDFATYYCQQGKTLPPTFGGGTKVEIK

SEQ ID NO: 2 - protein, 120 aa, human
QVQLVQSGAEVKKPGASVKVSCKASGYTFTGFWIHWVRQAPGQGLEWMGHINPGNGGTNYNEKFKRRVTMTRDKSISTAYMELSRLRSDDTAVYYCARSYSNYVRAMDYWGQGTLVTVSS

(19) United States
(12) Patent Application Publication
(10) Pub. No.: US 2015/0246117 A1
(43) Pub. Date: Sep. 3, 2015

(54) TREATMENT OF TYPE 2 DIABETES AND RELATED CONDITIONS

(71) Applicant: Ulf ERIKSSON, (US)

(72) Inventor: Ulf Eriksson, Stockholm (SE)

(21) Appl. No.: 14/430,263

(22) PCT Filed: Sep. 24, 2013
(86) PCT No.: PCT/IB2013/058822

§ 371 Date: Mar. 23, 2015

Related U.S. Application Data
(60) Provisional application No. 61/705,005, filed on Sep. 24, 2012.

(51) Int. Cl. A61K 39/395; A61K 45/06

(52) U.S. Cl. CPC A61K 39/3955; A61K 45/06

(57) ABSTRACT

The present disclosure provides methods of treating diabetic conditions, restoring insulin sensitivity and/or improving regulation of glycemia using an antagonist of VEGF-B, particularly in combination with other anti-diabetic agents such as insulin secretagogues. Compositions are also provided comprising one or more VEGF-B antagonists, particularly in combination with other anti-diabetic agents such as insulin secretagogues. In particular embodiments, the VEGF-B antagonist is an anti-VEGF-B antibody. In further particular embodiments, the other anti-diabetic agent or insulin secretagogue is a DPP-4 inhibitor or a GLP-1R agonist.

### BACKGROUND OF THE DISCLOSURE

Type 2 Diabetes (T2D) affects over 310 million individuals (WHO Media centre. World Health Organization Diabetes Fact sheet No. 312. Available online (2011)) worldwide and its prevalence is expected to increase significantly in the future (The Diabetes Prevention Program (DPP): description of lifestyle intervention. Diabetes Care 25, 2165-2171 (2002), Zimmet, P., et al. Nature 414, 782-787 (2001)). The disease is strongly associated with obesity and pathological lipid deposition within extra-adipose tissues (Stumvoll, M., et al. Lancet 365, 1333-1346 (2005)), and lipid-induced insulin resistance in muscles has been suggested to contribute to the development of diabetes. Excess lipid deposition in peripheral tissues impairs insulin sensitivity and glucose uptake, and has been proposed to contribute to the pathology of T2D (Perseghin, G., et al. Diabetes 48, 1600-1606 (1999), Samuel, V. T., et al. Lancet 375, 2267-2277 (2010), Unger, R. H. Annu Rev Med 53, 319-336 (2002)). Several mechanisms for the deleterious effect of ectopic lipids have been suggested, including the accumulation of diacylglycerols or ceramides that disrupt insulin signaling (Perseghin, G., et al. Diabetes 48, 1600-1606 (1999), Schmitz-Peiffer, C., et al. Diabetes 46, 169-178 (1997), Schmitz-Peiffer, C. Diabetes 59, 2351-2353 (2010)), and intra-cellular redistribution of key components of the molecular machinery required for glucose uptake (Bostrom, P., et al. Nat Cell Biol 9, 1286-1293 (2007), Bostrom, P., et al. Diabetes 59, 1870-1878 (2010)). Of the therapies currently available for the treatment of diabetes very few specifically target lipid deposition in peripheral tissues (Stumvoll, M., et al. Lancet 365, 1333-1346 (2005)).

T2D is the most common form of diabetes and is characterized by disorders of insulin action and insulin secretion, either of which may be the predominant feature. Both are usually present at the time that this form of diabetes is clinically manifested. Patients with T2D are characterized with a relative, rather than absolute, insulin deficiency and are resistant to the action of insulin. At least initially, and often throughout their lifetime, these individuals do not need insulin treatment to survive. T2D accounts for 90-95% of all cases of diabetes. This form of diabetes can go undiagnosed for many years because the hyperglycemia is often not severe enough to provoke noticeable symptoms of diabetes or symptoms are simply not recognized. The majority of patients with T2D are obese, and obesity itself may cause or aggravate insulin resistance. Many of those who are not obese by traditional weight criteria may have an increased percentage of body fat distributed predominantly in the abdominal region (visceral fat). Ketoacidosis is infrequent in this type of diabetes and usually arises in association with the stress of another illness. Whereas patients with this form of diabetes may have insulin levels that appear normal or elevated, the high blood glucose levels in these diabetic patients would be expected to result in even higher insulin values had their beta cell function been normal. Thus, insulin secretion is often defective and insufficient to compensate for the insulin resistance. On the other hand, some hyperglycemic individuals have essentially normal insulin action, but markedly impaired insulin secretion.

### BRIEF SUMMARY OF THE DISCLOSURE

The present invention provides methods of treating dyslipidemia, diabetic conditions, and restoring insulin sensitivity and/or improving regulation of glycemia using an antagonist of VEGF-B. The VEGF-B antagonist can be provided alone or in combination with other anti-diabetic agents such as insulin secretagogues. Compositions are also provided comprising one or more VEGF-B antagonists, alone or in combination with other anti-diabetic agents such as insulin secretagogues.

In particular embodiments, the VEGF-B antagonist is an anti-VEGF-B antibody. In a preferred embodiment, the anti-VEGF-B antibody is selected from the antibodies 1C6, 2F5, 2H10 and 4E12, and humanized, deimmunized or chimeric forms thereof, or anti-VEGF-B antibodies that compete for binding to VEGF-B with 2H10. In a particularly preferred embodiment, the anti-VEGF-B antibody is a humanized 2H10 mAb, or an antibody that has a binding affinity for human VEGF-B that is substantially equivalent to the binding affinity of mAb 2H10 for human VEGF-B. In a further preferred embodiment, the anti-VEGF-B antibody is a humanized 2H10 antibody comprising SEQ ID NO: 1 and/or SEQ ID NO: 2.

In embodiments of the combination treatments and compositions of the invention, the other anti-diabetic agent or insulin secretagogue that is combined with any VEGF-B antagonist as described above is a DPP-4 inhibitor or a GLP-1R agonist. In particular embodiments, the DPP-4 inhibitor is selected from vildagliptin, sitagliptin, saxagliptin, linagliptin, dutogliptin, gemigliptin, alogliptin, and berberine. In further particular embodiments, the GLP-1R agonist is selected from exenatide, liraglutide, CJC-1131, LY-307161, dulaglutide, CJC-1134, albiglutide, and taspoglutide.

### BRIEF DESCRIPTION OF THE FIGURES

FIG. 1. Pharmacological VEGF-B-neutralisation enhances glucose tolerance in diabetic rodent models. a-f, Analysis of control-treated or 2H10-treated pre-diabetic or diabetic db/db mice, as compared to lean mice. a-b, Blood glucose levels (n=10-16/group). c-d, Quantification of ORO staining in skeletal and cardiac muscles (n=4-6/group). e, IPGTT and AUC analysis (n=5-6/group). f, Plasma levels of TGs, HDL-c, LDL-c, NEFAs and ketones (n=8-12/group). g-h, Analysis of control-treated or 2H10-treated HFD-fed rats during a hyperinsulinaemic/euglycaemic clamp, as compared to control-treated normal diet (ND)-fed rats (n=5-6/group). g, Glucose infusion rate (GIR) and glucose disposal rate (GDR). h, [¹⁴C]-deoxyglucose uptake into skeletal and cardiac muscles. #/*P<0.05, ##/**P<0.01, ###/***P<0.001 compared to lean/db/db control mice (a-f), or control-treated ND-fed/HFD-fed rats (g-h). Values are means±s.e.m.

### DETAILED DESCRIPTION OF THE DISCLOSURE

This disclosure provides a novel combination therapy for treatment of diabetes, involving administration of a VEGF-B antagonist alone or in combination with an insulin secretagogue, such as a DPP-4 inhibitor and/or a GLP-1R agonist. This disclosure further provides compositions that include a VEGF-B antagonist, alone or in combination with an insulin secretagogue, such as a DPP-4 inhibitor and/or a GLP-1R agonist.

As used herein, the singular forms “a”, “an” and “the” include plural aspects unless the context clearly dictates otherwise. Thus, for example, reference to “an antibody” includes a single antibody, as well as two or more antibodies; reference to “a VEGF-B” includes a single VEGF-B as well as two or more VEGF-B molecules; reference to “an antagonist” includes a single antagonist as well as two or more antagonists; and so forth.

A “diabetic condition” as used herein includes Type 2 Diabetes, pre-diabetes and gestational diabetes. Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance in an oral glucose tolerance test (a 2 hour plasma glucose level of >140 mg/dl and <200 mg/dl); or insulin resistance (inability to respond to insulin activity), resulting in an increased risk of developing diabetes.

An “insulin secretagogue” is a substance that increases insulin secretion.

The present invention is directed to use of antagonists of vascular endothelial growth factor-B (VEGF-B) in treatment of diabetic conditions, alone or in combination with one or more insulin secretagogues.

VEGF-B acts on the vascular endothelium to regulate trans-endothelial transport of circulating fatty acids (FAs) into cardiac and skeletal muscle (Hagberg, C. E., et al. Nature 464, 917-921 (2010), Muoio, D. M. N Engl J Med 363, 291-293 (2010), Lahteenvuo, J. E., et al. Circulation 119, 845-856 (2009)). VEGF-B is expressed by tissue cells with a high metabolic activity, such as cardiac and skeletal myocytes, brown adipocytes and pancreatic β-cells, from where it signals to the endothelium (Hagberg, C. E., et al. Nature 464, 917-921 (2010), Olofsson, B., et al. Proc Natl Acad Sci USA 93, 2576-2581 (1996), Albrecht, I., et al. PLoS One 5, e14109). The interaction of VEGF-B with the endothelial receptor VEGF receptor 1 (VEGFR1) (Olofsson, B., et al. Proc Natl Acad Sci USA 95, 11709-11714 (1998)) and the co-receptor Neuropilin 1 (Makinen, T., et al. J Biol Chem 274, 21217-21222 (1999)) induces transcriptional upregulation of the vascular-specific Fatty Acid Transport Protein 3 (Fatp3) and the more ubiquitously expressed Fatp4 (Hagberg, C. E., et al. Nature 464, 917-921 (2010)). Mice lacking VEGF-B (Vegfb^{-/-}) (Aase, K., et al. Circulation 104, 358-364 (2001)) are healthy and fertile, but exhibit decreased FA uptake and lipid deposition in muscles.

A “VEGF-B antagonist” is a substance that can reduce or prevent VEGF-B activity. VEGF-B controls endothelial uptake and transport of fatty acids in heart and skeletal muscle. VEGF-B activities include activation of the endothelial receptor VEGF receptor 1 (VEGFR1) and the co-receptor Neuropilin 1, and induction of transcriptional upregulation of the Fatp3 and Fatp4 proteins. A “VEGF-B antagonist” administered according to the invention can restore insulin sensitivity, improve or normalize glucose tolerance, preserve pancreatic islet architecture, improve β-cell function and ameliorate dyslipidaemia. Thus, a VEGF-B antagonist inhibits or reduces diabetic symptoms and the diabetic condition.

A VEGF-B antagonist contemplated by the present invention may be an antibody which inhibits interaction between VEGF-B and VEGFR-1, an antisense compound which reduces VEGF-B expression, or an interfering nucleic acid which reduces VEGF-B expression. The preferred VEGF-B antagonists are antibodies that bind to VEGF-B and interfere with VEGF-B interaction with its receptor to inhibit the activity of VEGF-B (anti-VEGF-B antibodies); see for example U.S. Pat. No. 7,517,524.

Preferably, the anti-VEGF-B antibodies are monoclonal antibodies (“mAbs”) or antigen-binding fragments thereof. Even more preferably, the anti-VEGF-B antibodies are humanized antibodies including deimmunized or chimeric antibodies or human antibodies suitable for administration to humans. Humanized antibodies, prepared, for example, from murine monoclonal antibodies and human monoclonal antibodies which are prepared, for example, using transgenic mice or by phage display are particularly preferred. Antibodies in accordance with the present invention include humanized, deimmunized or chimeric forms of the murine monoclonal antibodies 1C6, 2F5, 2H10 and 4E12, and anti-VEGF-B antibodies that are suitable for administration to humans that compete for binding to VEGF-B with antibody 2H10. Antibody 2H10 is produced by the hybridoma deposited at the American Type Culture Collection (ATCC) as PTA-6889. Exemplary anti-VEGF-B antibodies useful in the practice of the invention are disclosed in U.S. Pat. No. 7,517,524; and in U.S. Patent Publication No. 20080260729, the contents of each of which are hereby incorporated by reference in their entirety.

In a preferred embodiment, the anti-VEGF-B antibody is a humanized 2H10 antibody (mAb 2H10), and in another preferred embodiment the anti-VEGF-B antibody is a humanized 2H10 antibody with an amino acid sequence comprising SEQ ID NO: 1 and/or SEQ ID NO: 2, such amino acid sequences comprising the variable light chain sequences and the variable heavy chain sequences shown in SEQ ID NOs: 29 and 30 as set out in US. Patent Publication No. 20080260729. A further preferred anti-VEGF-B antibody has a binding affinity for human VEGF-B that is substantially equivalent to (i.e., equal to or up to 3 times weaker) or stronger than the binding affinity of mAb 2H10 for human VEGF-B. Methods of determining binding affinities are known in the art. Preferred antibodies are those which bind human VEGF-B with a K_{D} value of 1×10⁻⁷ M or less; preferably 1×10⁻⁸ M or less; more preferably 1×10⁻⁹M or less; and most preferably 5×10⁻¹⁰ M or less as determined by surface plasmon resonance. One example of a biological activity of a mAb selected from 106, 2H10, 4E12 and 2F5 is its ability to bind to VEGF-B and inhibit signaling by VEGF-B through VEGFR-1.

This disclosure presents treatments and compositions comprising a VEGF-B antagonist. This disclosure further presents combination treatments and compositions comprising a VEGF-B antagonist in combination with an insulin secretagogue. In one embodiment, the insulin secretagogue is selected from a DPP-4 inhibitor and/or a GLP-1R agonist.

In a particular embodiment, this disclosure presents combination treatments and compositions including a VEGF-B antagonist in combination with an inhibitor of the enzyme dipeptidyl peptidase-4 (DPP-4). DPP-4, also known as CD26, is a serine protease that cleaves a dipeptide group from the N-terminal end of a number of proteins having at their N-terminal end a proline or alanine residue. DPP-4 substrates include the insulinotropic hormones Glucagon like peptide-1 (GLP-1) and Gastric inhibitory peptide (GIP). GLP-1 and GIP are active only in their intact forms; removal of their two N-terminal amino acids inactivates them. Inhibitors of DPP-4 prevent N-terminal degradation of GLP-1 and GIP, resulting in higher plasma concentrations of these hormones, increased insulin secretion and, therefore, improved glucose tolerance.

A “DPP-4 inhibitor” is a substance that can reduce or prevent DPP-4 activity, including reduction or prevention of serine protease activity, dipeptide cleavage activity, and inactivation of GLP-1 and/or GIP.

For example, DPP-4 inhibitors and their uses, particularly their uses in metabolic (especially diabetic) diseases, are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769 or WO2007/014886; or in WO 2004/050658, WO 2004/111051, WO 2005/058901 or WO 2005/097798; or in WO 2006/068163, WO 2007/071738 or WO 2008/017670; or in WO 2007/128721, WO 2007/128724 or WO 2007/128761, or WO 2009/121945. Several DPP-4 inhibitors that can be taken orally as a tablet have been developed.

Preferred DPP-4 inhibitors include vildagliptin (EU approved 2007, marketed in the EU by Novartis as GALVUS®), sitagliptin (marketed by Merck as JANUVIA®), saxagliptin (marketed as ONGLYZA®), linagliptin (FDA approved in 2011, marketed as TRAJENTA® by Eli Lilly Co and Boehringer Ingelheim), dutogliptin (Phenomix Corporation), gemigliptin (LG Life Sciences, Korea), alogliptin (Takeda Pharmaceutical Co.), and the herbal supplement berberine.

In a further particular embodiment, this disclosure further presents combination treatments and compositions including a VEGF-B antagonist in combination with a glucagon-like peptide-1 receptor (GLP-1R) agonist. GLP-1R belongs to the class B receptor sub-class of the G protein-coupled receptor (GPCR) superfamily that regulates many important physiological and pathophysiological processes. GLP-1R is a seven-transmembrane spanning receptor coupled to G-protein activation, increased cAMP production and activation of PKA. Other responses to the actions of GLP-1 include, for example, pancreatic β-cell proliferation and expansion concomitant with a reduction of β-cell apoptosis. In addition, GLP-1 activity can result in increased expression of the glucose transporter-2 (GLUT2) and glucokinase genes in pancreatic cells.

A “GLP-1 receptor agonist” or a “GLP-1R agonist” is a molecule that increases the amount of activation of GLP-1R, producing effects similar to those produced by the naturally-occurring agonists GLP-1 and exendin-4. GLP-1R agonists increase the activation of GLP-1R, e.g., by binding to and activating GLP-1R, by causing a conformational change in the GLP-1R, by causing activation of a G protein coupled to the GLP-1R, by causing GLP-1R to remain in an activated (e.g., in the active conformation) condition for a longer period of time (including indefinitely), by mimicking the binding of naturally-occurring agonists, by modulating the binding of naturally-occurring agonists, by blocking inhibitors of GLP-1 or otherwise modulating GLP-1R activation or initiating the cascade of intracellular events that is characteristic of GLP-1R activation. Preferred GLP-1R agonists include GLP-1 analogues or mimetics, and GLP-1 receptor agonists, such as exendin-4 (exenatide), liraglutide (N,N-2211), CJC-1131, LY-307161, dulaglutide (LY2189265), CJC-1134 (exendin-4-albumin conjugate), albiglutidc (GSK716155; albugon), taspoglutidc, and those disclosed in WO 00/42026 and WO 00159887.

GLP-1 (7-36) amide is not very useful for treatment of T2D, since it must be administered by continuous subcutaneous infusion. Several long-lasting analogs having insulinotropic activity have been developed, and two, exenatide (BYETTA®) and liraglutide (VICTOZA®), have been approved for use in the U.S.

The combination therapeutic treatments include administration of an effective amount of a VEGF-B antagonist prior to, concurrently with, or subsequent to administration of an effective amount of the insulin secretagogue. The insulin secretagogue can be a DPP-4 inhibitor, a GLP-1R agonist, or a combination of the two. In a preferred embodiment, the VEGF-B antagonist is an anti-VEGF-B antibody. In another preferred embodiment the VEGF-B antagonist is a humanized 2H10 antibody or anti-VEGF-B antibodies that is suitable for administration to humans and that competes with antibody 2H10 for binding to VEGF-B. In another preferred embodiment, the anti-VEGF-B antibody is a humanized 2H10 antibody, and in another preferred embodiment the anti-VEGF-B antibody is a humanized 2H10 antibody comprising SEQ ID NO: 1 and/or SEQ ID NO: 2 as set out in SEQ ID NOs: 29 and 30 of US. Patent Publication No. 20080260729.

Combination compositions include a VEGF-B antagonist and an insulin secretagogue selected from a DPP-4 inhibitor and a GLP-1R agonist in a pharmaceutically acceptable carrier.

Examples of combination compositions include a combination of a VEGF-B antagonist selected from the humanized antibodies 1C6, 2F5, 2H10, 4E12, or an anti-VEGF-B antibody that competes for binding to VEGF-B with antibody 2H10, and an insulin secretagogue selected from a DPP-4 inhibitor and a GLP-1R agonist. In one embodiment, the combination composition includes a humanized VEGF-B antagonist antibody, such as humanized 1C6, 2F5, 2H10, or 4E12, in combination with a DPP-4 inhibitor and/or an GLP-1R agonist.

In a preferred embodiment, the combination composition includes a humanized 2H10 antibody (mAb 2H10), particularly a humanized 2H10 antibody with an amino acid sequence comprising SEQ ID NO: 1 and/or SEQ ID NO: 2 as set out in SEQ ID NOs: 29 and 30 of US. Patent Publication No. 20080260729, in combination with a DPP-4 inhibitor selected from vildagliptin, sitagliptin, saxagliptin, linagliptin, dutogliptin, gemigliptin, berberine, and similar DPP-4 inhibitors or analogs thereof. Thus, exemplary combinations include 2H10 antibody and vildagliptin, 2H10 antibody and sitagliptin, 2H10 antibody and saxagliptin, 2H10 antibody and linagliptin, 2H10 antibody and dutogliptin, 2H10 antibody and gemigliptin, 2H10 antibody and berberine, and similar combinations.

In another embodiment, the combination composition includes a humanized 2H10 antibody and a DPP-4 inhibitor as disclosed in any of WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769 or WO2007/014886; or in WO 2004/050658, WO 2004/111051, WO 2005/058901 or WO 2005/097798; or in WO 2006/068163, WO 2007/071738 or WO 2008/017670; or in WO 2007/128721, WO 2007/128724 or WO 2007/128761, or WO 2009/121945.

In another preferred embodiment, the combination composition includes a humanized 2H10 antibody (mAb 2H10), particularly a humanized 2H10 antibody with an amino acid sequence comprising SEQ ID NO: 1 and/or SEQ ID NO: 2, in combination with a GLP-1R agonist selected from a GLP-1 analogue or mimetic thereof, a GLP-1 receptor agonist, such as exendin-4/exenatide, liraglutide, CJC-1131, LY-307161, dulaglutide, CJC-1134, albiglutide/albugon, taspoglutide, or analogs thereof, or a GLP-1 receptor agonist as disclosed in WO 00/42026 or WO 00/59887. Thus, exemplary combinations include 2H10 antibody and exendin-4/exenatide, 2H10 antibody and liraglutide, 2H10 antibody and CJC-1131, 2H10 antibody and LY-307161, 2H10 antibody and dulaglutide, 2H10 antibody and CJC-1134, 2H10 antibody and albiglutide/albugon, 2H10 antibody and taspoglutide, and similar combinations.

The terms “effective amount” and “therapeutically effective amount” of an agent as used herein mean a sufficient amount of the VEGF-B antagonist to provide the desired therapeutic or physiological effect or outcome including reducing diabetic symptoms and improving undesirable effects, e.g. side effects, are sometimes manifested along with the desired therapeutic effect; hence, a practitioner balances the potential benefits against the potential risks in determining what is an appropriate “effective amount”. The exact amount of agent required may vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. The ability of a VEGF-B antagonist, preferably an anti-VEGF-B antibody, to treat diabetic conditions can be evaluated in an animal model system predictive of efficacy in human diabetic treatment. One of ordinary skill in the art would be able to determine such amounts based on such factors as the subject's size, the severity of the subject's symptoms, and the particular composition or route of administration selected.

By “pharmaceutically acceptable” carrier and/or diluent is meant a pharmaceutical vehicle comprised of a material that is not biologically or otherwise undesirable, i.e. the material may be administered to a subject along with the selected active agent without causing any or a substantial adverse reaction. Carriers may include any and all solvents, dispersion media, coatings, antibacterial and antifungal agents, agents used for adjusting tonicity, buffers, chelating agents, and absorption delaying agents and the like.

Similarly, a “pharmacologically acceptable” salt, ester, amide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of diabetes, elimination of symptoms of diabetes, prevention of the occurrence of symptoms of diabetes and/or their underlying cause and improvement or remediation or amelioration of damage following onset of diabetes.

Treatment of diabetes refers to the administration of a compound or combination described herein to treat a diabetic condition. One outcome of the treatment of diabetes is to reduce an increased plasma glucose concentration. Another outcome of the treatment of diabetes is to reduce an increased insulin concentration. Still another outcome of the treatment of diabetes is to reduce an increased blood triglyceride concentration. Still another outcome of the treatment of diabetes is to increase insulin sensitivity. Still another outcome of the treatment of diabetes may be enhancing glucose tolerance in a subject with glucose intolerance. Still another outcome of the treatment of diabetes is to reduce insulin resistance. Another outcome of the treatment of diabetes is to normalize plasma insulin levels. Still another outcome of treatment of diabetes is an improvement in glycemic control, particularly in type 2 diabetes. Yet another outcome of treatment is to increase hepatic insulin sensitivity.

A “subject” as used herein refers to an animal, preferably a mammal and more preferably a human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient as well as subject. The compounds and methods of the present invention have applications in human medicine and veterinary medicine.

The humanised mouse monoclonal antibody targeting VEGF-B (2H10) acts to reduce the accumulation of fatty acids within tissues and, as a consequence, restores insulin sensitivity and the regulation of glycemia. The mAb can be used in patients with T2D whose disease is not well controlled by first line medication, such as metformin. It is likely that these patients will have been transitioned onto treatment with either sulfonylureas, or to classes of compounds known as DPP-4 inhibitors or GLP-1R agonists. These latter compounds act to increase insulin production and while they are well tolerated their effectiveness can be modest at best. The mechanism of action (MOA) of a VEGF-B antagonist and of a DPP-4 inhibitor or GLP-1R agonist is complimentary and a VEGF-B antagonist would enhance and extend the benefits of the DPP-4 inhibitors/GLP-1R agonists.

The disclosure herein shows that decreased VEGF-B signaling in rodent models of T2D restored insulin sensitivity and improved glucose tolerance. Genetic deletion of Vegfb in diabetic db/db mice prevented ectopic lipid deposition, increased muscle glucose uptake and maintained normoglycaemia. Pharmacological inhibition of VEGF-B signaling by antibody administration to db/db mice enhanced glucose tolerance, preserved pancreatic islet architecture, improved β-cell function and ameliorated dyslipidaemia, key elements of T2D and the metabolic syndrome. The potential use of VEGF-B-neutralisation in T2D was further elucidated in rats fed a high fat diet, where it normalised insulin sensitivity and increased glucose uptake to skeletal muscle and heart. These results demonstrate that the vascular endothelium can function as an efficient barrier towards excess muscle lipid uptake even under conditions of severe obesity and T2D, and that this barrier can be maintained by inhibition of VEGF-B signaling.

Mice fed a high fat diet (HFD mice) and diabetic db/db mouse models are characterised by obesity, hyperglycaemia and ectopic lipid deposition. Vegfb-deletion in these mouse models resulted in decoupling of mitochondrial gene expression was from the expression of Vegfb and its downstream target Fatp3 in obese mice. Expression of mitochondrial genes is reduced in HFD and db/db mice, while skeletal and cardiac muscle expression of Vegfb and Fatp3 is increased. Thus, targeting VEGF-B-mediated FA-uptake has beneficial effects on intra-muscular lipid accumulation and diabetic phenotypes.

The inventors have determined that, although Vegfb^{-/-} mice fed a HFD (HFD-Vegfb^{-/-} mice) gained more weight and had larger fat pads than HFD-wt mice, they showed lower blood glucose levels. These effects were even more prominent in Vegfb^{-/-} mice crossed with db/db mice (db/db//Vegfb^{-/-} mice). Db/db///Vegfb^{-/-} mice had normal blood glucose levels in both the fasted and postprandial state, in contrast to the severely hyperglycaemic db/db control animals. The db/db//Vegfb^{-/-} and db/db//Vegfb^{-/-} mice had equal or lower plasma insulin levels compared to db/db controls, indicating that their normalised blood glucose was not due to compensatory hyperinsulinaemia. All three genotypes had similar body weight and weight gain until 20 weeks of age, when the db/db control animals became catabolic, i.e. stopped gaining weight and developed glucosuria. In contrast, db/db mice lacking one or both Vegfb-alleles did not become catabolic. Deletion of Vegfb did not affect food intake in mice on either a normal diet or HFD, or in diabetic mice.

Deletion of Vegfb in db/db mice reduced Fatp3 mRNA expression and significantly decreased lipid deposition in skeletal muscle and myocardium. VEGF-B depletion also reduced the accumulation of triglycerides (TGs) in isolated pancreatic islets from db/db//Vegfb^{+/-} and db/db//Vegf^{-/-} mice, but did not affect lipid accumulation in liver. Uptake and accumulation of the glucose analogue [2-¹⁸F]-2-Fluoro-2-deoxy-D-glucose ([¹⁸F]FDG) was assessed in db/db and db/db//Vegfb^{-/-} mice using positron emission tomography (PET). Analyses of the db/db//Vegfb^{-/-} mice showed a 29% increase in cardiac [¹⁸F]FDG-uptake as compared to db/db controls. Post-PET measurement of radioactivity in dissected tissues confirmed increased glucose uptake to both skeletal and cardiac muscles in the db/db//Vegfb^{-/-} mice. Taken together, the deletion of either one or both copies of Vegfb in diabetic mouse models was sufficient to reduce peripheral lipid deposition in muscular tissues, increase glucose uptake and maintain normoglycaemia.

T2D and the metabolic syndrome are characterised by glucose intolerance, insulin resistance and dyslipidaemia. HFD-Vegfb^{-/-} mice demonstrated improved glucose tolerance compared to HFD-wt mice, and were comparable to lean mice in their response to insulin injections. Similarly, db/db//Vegfb^{-/-} and db/db//Vegfb^{+/-} mice were more glucose tolerant and insulin sensitive than db/db controls. The expression of gluconeogenic and lipogenic genes was increased in the livers of control db/db mice, most likely due to hepatic insulin resistance (Ref 6), but the db/db//Vegfb^{-/-} mice exhibited a normal hepatic expression pattern of such genes. Furthermore, dyslipidemia was ameliorated in the db/db//Vegfb^{-/-} mice. The db/db//Vegfb^{-/-} mice had reduced plasma triglyceride levels and LDL/VLDL-bound cholesterol (LDL-c) levels, and higher levels of HDL-bound cholesterol (HDL-c) than the db/db controls. The db/db//Vegfb^{-/-} mice also had a normal HDL-c to LDL-c ratio (lean mice 4.3±0.5 (a.u.); db/db 1.8±0.3; db/db//Vegfb^{-/-} 4.0±0.4; db/db//Vegfb^{-/-} 4.2±0.6), indicating a lower risk for cardiovascular disease, although total plasma cholesterol was higher in db/db//Vegfb^{-/-} mice compared to lean mice. Moreover, the levels of circulating non-esterified FAs (NEFAs) and ketones were reduced in db/db//Vegfb^{-/-} mice. Genetic deletion of Vegfb thus targeted several hallmarks of T2D and improved the metabolic balance.

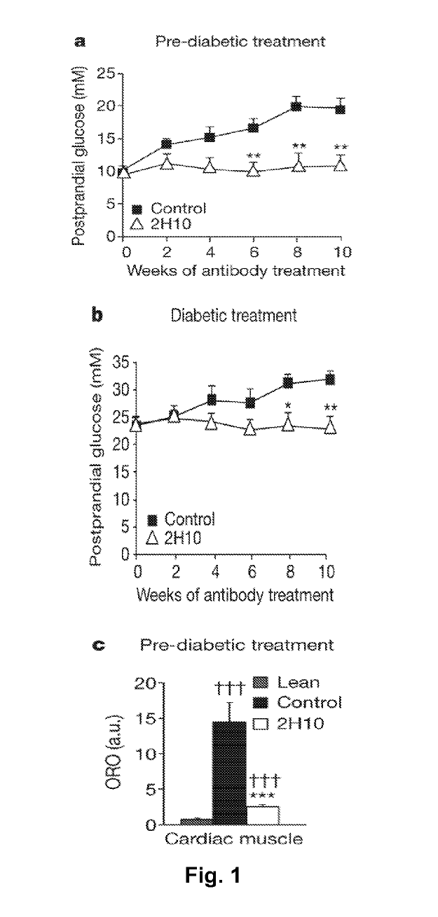

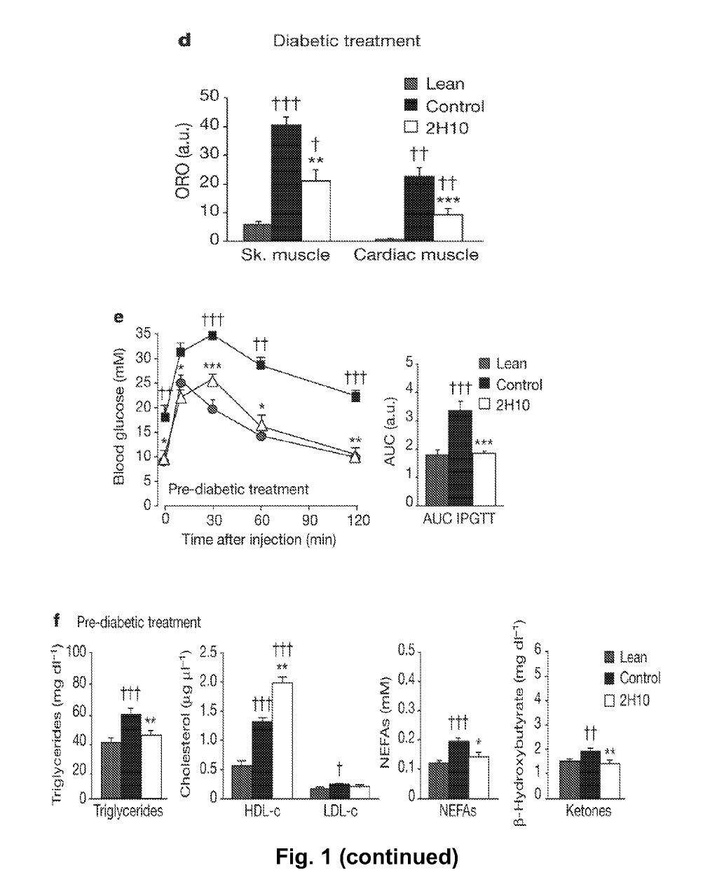

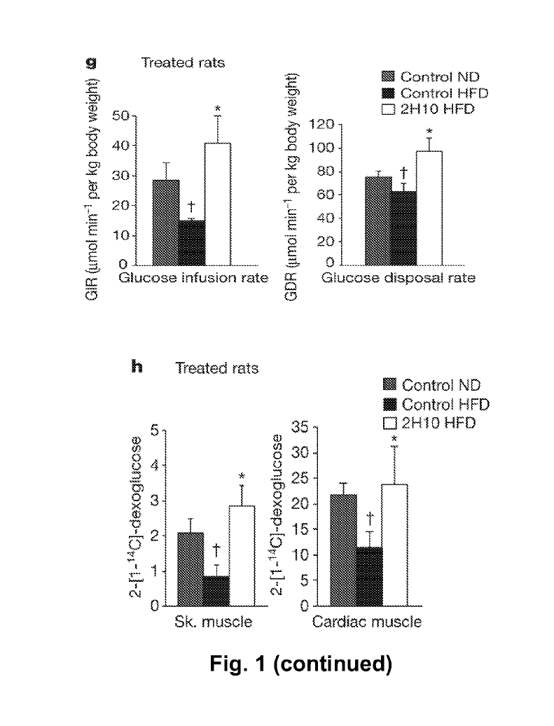

To investigate if pharmacological inhibition of VEGF-B signaling could ameliorate T2D and the metabolic syndrome, db/db mice were injected with a specific neutralizing anti-VEGF-B antibody (2H10, mouse version (Scotney, P. D., et al. Clin Exp Pharmacol Physiol 29, 1024-1029 (2002) and U.S. Pat. No. 7,517,524)), or a control antibody for 10 weeks. Two treatment strategies were applied, one preventive using pre-diabetic db/db mice, and one therapeutic using diabetic db/db mice. Both strategies lowered cardiac Fatp3 mRNA levels at the end of the study, whereas no effect on Vegfb-expression was detected. Treatment of pre-diabetic db/db mice with 2H10 prevented the development of hyperglycaemia (FIG. 1a), and the progression of hyperglycaemia was halted in diabetic db/db receiving 2H10 (FIG. 1b). At the end of the study, db/db mice receiving 2H10 displayed reduced lipid deposition in skeletal muscle and heart (FIG. 1c-d), and improved glucose tolerance compared to control-treated db/db mice (FIG. 1e), while no effect on weight-gain was observed. Administration of 2H10 protected the pre-diabetic db/db mice against elevated levels of circulating TGs, LDL-c, NEFAs and ketones, and increased plasma HDL-c levels (FIG. 1f). 2H10-treatment of the diabetic db/db mice had no effect on plasma TG levels, and increased the levels of both HDL-c and LDL-c as compared to the controls. However, 2H10-treatment did lower both circulating NEFAs and ketones in the diabetic db/db mice. In summary, anti-VEGF-B treatment over a 10 week period, in either a preventative or a therapeutic context, was able to significantly limit disease progression.

To further determine the therapeutic potential of an anti-VEGF-B antibody in insulin resistance and T2D, rats were fed a HFD (HFD-rats) for 8 weeks, and in parallel given 2H10 (chimeric mouse/rat antibody), or a control antibody. Treatment with 2H10 normalised glucose tolerance and glucose-stimulated insulin secretion in the HFD-rats. A hyperinsulinacmic/euglycaemic clamp study demonstrated that control-treated HFD-rats exhibited reduced glucose infusion rates (GIR) and glucose disposal rates (GDR) compared to lean control-treated animals, indicative of insulin resistance (FIG. 1g). In contrast, 2H10-treatment of the HFD-rats increased both GIR and GDR, and significantly promoted glucose uptake into skeletal muscle and heart (FIG. 1g-h). Food consumption and endogenous glucose production remained unchanged in the 2H10-treated HFD-rats compared to HFD controls. Anti-VEGF-B treatment thus restored insulin sensitivity, increased muscle glucose usage, and reversed the T2D pathology in these rodent models.

The improved glucose tolerance after VEGF-B-neutralisation in mice and rats suggested that inhibition of VEGF-B might preserve islet function in addition to ameliorating insulin resistance. Histochemical analysis showed that 2H10-treatment of pre-diabetic and diabetic mice did not prevent pancreatic islet hyperplasia or influence islet density. However, 2H10-treated diabetic mice had large islets with few signs of degeneration, whereas the diabetic control mice had small islets with grossly disrupted architecture. The 2H10-treated db/db mice had normalised expression of insulin and glucagon within their islets as compared to control-treated mice. Furthermore, 2H10-treated animals had a normalised ratio between glucagon and insulin staining, and less centrally located β-cells within the islets, indications of preserved islet integrity. Staining for the apoptosis marker cleaved caspase 3 confirmed that the db/db mice treated with 2H10 displayed significantly reduced β-cell apoptosis. Thus, therapeutic inhibition of VEGF-B signaling preserved insulin production and islet functionality by protecting islet architecture and preventing β-cell apoptosis.

Insulin resistance characterises >90% of T2D patients (Ref 27). However, with exception of the thiazolidinediones, none of the current treatment options primarily targets this aspect of T2D pathology. Although thiazolidinediones have recently been linked to adverse cardiovascular outcomes, the use of these drugs provided a clear proof-of-concept that lowering peripheral lipid deposition can ameliorate T2D (Chaggar, P. S., et al. Diab Vasc Dis Res 6, 146-152 (2009)). The inventors have shown, in four different rodent models of insulin resistance and T2D, that glucose intolerance can be efficiently reduced by targeting VEGF-B-regulated FATP expression and endothelial lipid transport.

Anti-VEGF-B treatment also improved pancreatic β-cell function, possibly via reducing TG accumulation within the islets. Direct effects of VEGF-B signaling on β-cells are unlikely as VEGFR1 is only expressed by endothelial cells in the pancreas (Ref 16). The inventors have studied HFD-fed mice over-expressing VEGF-B selectively in the pancreatic β-cells (RIP-VEGF-B mice). HFD-fed RIP-VEGF-B mice were indistinguishable from wt mice in glucose tolerance, insulin sensitivity, islet morphology and circulating levels of insulin. Furthermore, no alterations were detected in lean RIP-VEGF-B mice regarding pancreatic islet physiology, lipid staining or Fatp-expression (Ref 16). Hence, systemic 2H10-treatment protects the islets via a combination of reduced lipotoxicity, and improved peripheral insulin sensitivity leading to a reversal of hyperglycaemia and thereby no requirement for hyperinsulinemia.

In summary, both genetic and pharmacological inhibition of VEGF-B-signaling can limit ectopic lipid accumulation of FAs, restore peripheral insulin sensitivity and muscle glucose uptake, and preserve pancreatic functionality. Inhibition of VEGF-B signaling represents a novel and promising treatment option that targets the underlying pathology of T2D, thereby improving several adverse metabolic consequences of the disease.

In view of the observed effects of a VEGF-B antagonist in models of T2D they may be particularly effective in a method of treating diabetic conditions when used in combination with a DPP-4 inhibitor or a GLP-1R agonist. The combination may allow dosing with lower amounts of the drugs than would be necessary if either drug were used alone, which can reduce side effects or the risk of side effects (see Table 1). Additionally, the combination may allow reduced frequency of dosing, may result in reduced effects on concomitantly administered drugs and may potentially lead to treatment of additional patient populations.

For example, the combination of a VEGF-B antagonist with a DPP-4 inhibitor or a GLP-1R agonist can reduce the amount of DPP-4 inhibitor or GLP-1R agonist that is administered to a patient by 10%, 20%, 30%, 40%, 50%, 60%, 70%, 80%, 90% or 100%.

Lowering the dose of DPP-4 inhibitor can reduce the incidence and/or severity of commonly seen side effects in patients treated with DPP4 inhibitors, including upper respiratory tract infection, nasopharyngitis, urinary tract infection and headache. Furthermore, treating patients with lower amounts of DPP-4 inhibitors can reduce the risk of hypoglycaemia, pancreatitis, hepatic dysfunction, impaired renal function, hypersensitivity reactions (e.g. anaphylaxis, angioedema, exfoliative skin conditions) and peripheral edema, which have been reported in patients treated with DPP-4 inhibitors. See, Pattzi et al (2010) Diabetes Obes Metab 12:348-355; Lim et al (2008) Clin Ther 30: 1817-30; Andukuri et al (2009) Diabetes, Metabolic Syndrome and Obesity: Targets and Therapy 2: 117-126; Yin et al (2008) Metabolism 57: 712-717; Zhao Y N et al (2004) American journal of Chinese Medicine 32: 921-929.

Lowering the dose of DPP-4 inhibitor can also minimise the impact of these drugs on other concomitantly administered drugs, e.g. thiazides, corticosteroids, thyroid products, sympathomimetics, digoxin, CYP3A4/5 inhibitors (e.g., ketoconazole) and P-glycoprotein/CYP3A4 inducer (e.g. rifampin).

Exemplary DPP-4 inhibitors contemplated for combination therapy according to the disclosed methods, side effects associated with such inhibitors, and drug interactions associated with the inhibitors, that can be reduced by combination therapy with a VEGF-B antagonist, are provided in Table 1:

TABLE 1
Exemplary DPP-4 inhibitors and side effects
DPP-4 inhibitors

| | Dosing route, | | | |
|---|---|---|---|---|
| | amount, | | | |
| Drug name | frequency, | | | Contraindications/ |
| Marketed/ | terminal half- | Warnings and precautions/adverse | | specific patient |
| developed by | life | reactions | Drug interactions | populations |
| GALVUS ® (EU) | Oral | Rare cases of hepatic dysfunction | Hypoglycaemic | Safety and effectiveness |
| (vildagliptin) | 100 mg (50 mg | (including hepatitis), angioedema and | effect of | in children under 18 |
| Novartis | twice | pancreatitis have been reported. | vildagliptin may be | years have not been |
| Pharma GmbH | daily) as | Patients receiving vildagliptin in | reduced by certain | established. |
| | monotherapy | combination with a sulphonylurea | active substances, | Pregnancy: studies in |
| | 50 mg once | may be at risk for hypoglycaemia. | including thiazides, | animals have shown |
| | daily in dual | GALVUS: EPAR—Product Information | corticosteroids, | reproductive toxicity at |
| | combination | (Last updated 19 Aug. 2013), | thyroid products | high doses. Due to lack |
| | with a | European Medicines Agency website. | and | of human data, should |
| | sulphonylurea | | sympathomimetics | not be used during |
| | Half-life: | | | pregnancy. |
| | 2 h after i.v.; | | | Breast-feeding: animal |
| | 3 h after oral | | | studies have shown |
| | | | | excretion of vildagliptin |
| | | | | in milk. Should not be |
| | | | | used during breast- |
| | | | | feeding. |
| | | | | Fertility: no studies on |
| | | | | the effect on human |
| | | | | fertility have been |
| | | | | conducted. |
| JANUVIA | Oral | Adverse reactions reported in ≧5% of | Digoxin | Safety and effectiveness |
| (sitagliptin) | 100 mg once | patients treated with JANUVIA and | There was a slight | of JANUVIA in children |
| Merck Sharp | daily | more commonly than in patients | increase in the | under 18 years have not |
| Dohme | Half-life: | treated with placebo are: upper | area under the | been established |
| | 12.4 h after | respiratory tract infection, | curve (AUC, 11%) | In animal studies, |
| | oral | nasopharyngitis and headache. | and mean peak | JANUVIA did not impair |
| | | There have been postmarketing | drug concentration | fertility or harm the |
| | | reports of acute pancreatitis, | (Cmax, 18%) of | fetus. However, there |
| | | worsening renal function and serious | digoxin with the | are no adequate and |
| | | hypersensitivity reactions in patients | co-administration | well-controlled studies |
| | | treated with JANUVIA. The | of 100 mg | in pregnant women. |
| | | hypersensitivity reactions include | sitagliptin for 10 | |
| | | anaphylaxis, angioedema, and | days. Patients | |
| | | exfoliative skin conditions including | receiving digoxin | |
| | | Stevens-Johnson syndrome. | should be | |
| | | There is an increased risk of | monitored | |
| | | hypoglycemia when JANUVIA is added | appropriately. No | |
| | | to an insulin secretagogue (e.g., | dosage adjustment | |
| | | sulfonylurea) or insulin therapy. | of digoxin or | |
| | | Reference: Label dated 19 Aug. | JANUVIA is | |
| | | 2013, US Food and Drug | recommended. | |
| | | Administration (FDA) website. | | |
| ONGLYZA | Oral | There have been postmarketing | Strong CYP3A4/5 | No adequate and well- |
| (saxagliptin) | 2.5 mg or | reports of acute pancreatitis, serious | inhibitors (e.g., | controlled studies in |
| Bristol-Myers | 5 mg once | hypersensitivity reactions (including | ketoconazole): | pregnant women. |
| Squibb | daily | anaphylaxis, angioedema, and | Coadministration | Safety and effectiveness |
| and Astra | Half-life: | exfoliative skin conditions) in patients | with ONGLYZA | of ONGLYZA in pediatric |
| Zeneca | 2.5 h after | taking ONGLYZA. | significantly | patients under 18 years |
| | oral 5 mg; and | Hypoglycemia: In add-on to | increases | of age have not been |
| | 3.1 h (active | sulfonylurea, add-on to insulin, and | saxagliptin | established. |
| | metabolite) | add-on to metformin plus | concentrations. | |
| | | sulfonylurea trials, confirmed | Recommend | |
| | | hypoglycemia was more common in | limiting ONGLYZA | |
| | | patients treated with ONGLYZA | dosage to 2.5 mg | |
| | | compared to placebo. | once daily. | |
| | | Adverse reactions reported in ≧5% of | | |
| | | patients treated with ONGLYZA and | | |
| | | more commonly than in patients | | |
| | | treated with placebo are upper | | |
| | | respiratory tract infection, urinary | | |
| | | tract infection, and headache. | | |
| | | Peripheral edema was reported more | | |
| | | commonly in patients treated with | | |
| | | the combination of ONGLYZA and a | | |
| | | thiazolidinedione (TZD) than in | | |
| | | patients treated with the combination | | |
| | | of placebo and TZD. | | |
| | | Reference: Label dated 24 May 2013, | | |
| | | US FDA website. | | |
| TRADJENTA | Oral | There have been postmarketing | P- | Pregnancy: There are no |
| (linagliptin) | 5 mg once | reports of acute pancreatitis, | glycoprotein/CYP3 | adequate and well- |
| Eli Lilly Co and | daily | including fatal pancreatitis. | A4 inducer: The | controlled studies in |
| Boehringer | Half-life: | Adverse reactions reported in ≧5% of | efficacy of | pregnant women. |
| Ingelheim | >100 h after 5 mg | patients treated with TRADJENTA and | TRADJENTA may | Nursing mothers: |
| | dose | more commonly than in patients | be reduced when | Caution should be |
| | | treated with placebo included | administered in | exercised when |
| | | nasopharyngitis. | combination (e.g., | TRADJENTA is |
| | | Hypoglycemia was more commonly | with rifampin). Use | administered to a |
| | | reported in patients treated with the | of alternative | nursing woman |
| | | combination of TRADJENTA and | treatments is | Pediatric patients: |
| | | sulfonylurea compared with those | strongly | Safety and effectiveness |
| | | treated with the combination of | recommended. | of TRADJENTA in |
| | | placebo and sulfonylurea | | patients below the age |
| | | Reference: Label dated 18 Jun. 2013, | | of 18 have not been |
| | | US FDA website. | | established |
| dutogliptin | Oral | In August 2010, Phenomix terminated | | |
| (PHX1149T) | 100-400 mg | multiple clinical trials. No reason was | | |
| Phenomix | once daily | given. | | |
| Corporation | Half-life: | | | |
| | 10-13 h | | | |
| Gemigliptin | Oral | No serious adverse events recorded. | | |
| (LC15-0444) | 25-600 mg | | | |
| LG Life | Half-life: | | | |
| Sciences, | 17-21 h | | | |
| Korea | | | | |
| Double-Crane | | | | |
| Pharmaceutical | | | | |
| Co. (DCPC) | | | | |
| Alogliptin | Half-life: | The agent is relatively well tolerated | | |
| Takeda | 12.4-21.4 h | with few adverse effects, the major | | |
| Pharmaceutical | | finding being a marginally higher rate | | |
| Co. | | of skin events, primarily pruritus. | | |
| berberine | 500 mg 3x | Transient gastrointestinal adverse | | |
| | daily | effects | | |
| | Half-life: | | | |
| | 3-4 h | | | |

Lowering the dose of GLP-1R agonist can reduce the incidence and/or severity of commonly seen side effects in patients treated with GLP-1R agonists, including nausea, hypoglycemia, vomiting, diarrhea, feeling jittery, dizziness, headache, dyspepsia, constipation, asthenia, injection site pruritis, injection site nodule, dyspepsia and anti-drug antibodies (e.g. anti-liraglutide antibodies). Furthermore, treating patients with lower amounts of GLP-1R agonists can reduce the risk of hypoglycaemia, pancreatitis, impaired renal function and hypersensitivity reactions (e.g. anaphylaxis, angioedema), which have been reported in patients treated with GLP-1R agonists. See, Kim et al (2003) Diabetes 52:751-9; Baggio et al (2008) Gastroenterology 134:1137-47; Baggio et al (2004) Diabetes 53:2492-500; Rosenstock et al (2013) Diabetes Care 36:498-504. A lower dose of GLP-1R agonist can also reduce the potential risk of medullary thyroid carcinoma in patients treated with, for example, exenatide or liraglutide.

Lowering the dose of GLP-1R agonist can also minimise the impact of these drugs on other concomitantly administered drugs, e.g. orally administered medications and warfarin.

Exemplary GLP-1R agonists contemplated for combination therapy according to the disclosed methods, side effects associated with such agonists, and drug interactions associated with the agonists, that can be reduced by combination therapy with a VEGF-B antagonist, are provided in Table 2:

TABLE 2
Exemplary GLP-1R agonists and side effects
GLP-1R agonists

| | Dosing route, | | | Contraindications/ |
|---|---|---|---|---|
| | amount, | | | specific |
| | frequency, | Warnings and precautions/adverse | | patient |
| Drug name | drug half-life | reactions | Drug interactions | populations |
| BYETTA (US) | Subcutaneous | Postmarketing reports of pancreatitis; | May impact | Pregnancy: |
| Exendin-4 | 5-10 | hypersensitivity reactions (e.g. anaphylaxis | absorption of orally | Based on animal |
| (exenatide) | micrograms | and angioedema), renal impairment. | administered | data, BYETTA |
| Amylin and Eli | twice daily | Increased risk of hypoglycemia when used in | medications | may cause fetal |
| Lilly and Co | | combination insulin or an insulin | Warfarin: | harm |
| | | secretagogue. | Postmarketing | Nursing |
| | | Not recommended in patients with severe | reports of increased | Mothers: |
| | | gastrointestinal disease (e.g., gastroparesis). | international | Caution should |
| | | Most common (≧5%) and occurring more | normalized ratio | be exercised |
| | | frequently than placebo in clinical trials: | (INR) sometimes | when BYETTA is |
| | | nausea, hypoglycemia, vomiting, diarrhea, | associated with | administered to |
| | | feeling jittery, dizziness, headache, | bleeding. | a nursing woman |
| | | dyspepsia, constipation, asthenia. Nausea | | |
| | | usually decreases over time | | |
| | | Reference: | | |
| | | Label dated 19 Oct. 2011, US FDA | | |
| | | website. | | |
| BYDUREON ™ | Subcutaneous | Based on animal data, there may be a risk of | May impact | Pregnancy: may |
| Exendin-4 | 2 mg once | medullary thyroid carcinoma. | absorption of orally | cause fetal harm |
| (exenatide) | weekly | Postmarketing reports of pancreatitis; | administered | Nursing |
| Bydureon is | | hypersensitivity reactions (e.g. anaphylaxis | medications | Mothers: |
| an extended- | | and angioedema). | Warfarin: | Caution should |
| release | | Increased risk of hypoglycemia when used in | Postmarketing | be exercised |
| formulation of | | combination insulin or an insulin | reports of increased | when |
| exenatide | | secretagogue. | international | administered to |
| Amylin | | Not recommended in patients with severe | normalized ratio | a nursing woman |
| | | gastrointestinal disease (e.g., gastroparesis). | (INR) sometimes | Do not use if |
| | | Most common (≧5%) and occurring more | associated with | personal or |
| | | frequently than comparator in clinical trials: | bleeding. | family history of |
| | | nausea, diarrhea, headache, vomiting, | | medullary |
| | | constipation, injection site pruritus, injection | | thyroid |
| | | site nodule, and dyspepsia | | carcinoma or in |
| | | Label dated 27 Jan. 2013, US FDA | | patients with |
| | | website. | | Multiple |
| | | | | Endocrine |
| | | | | Neoplasia |
| | | | | syndrome type 2 |
| | | | | Do not use if |
| | | | | history of serious |
| | | | | hypersensitivity |
| | | | | to exenatide or |
| | | | | any product |
| | | | | components |
| VICTOZA ® | Subcutaneous | Based on animal data, there may be a risk of | Victoza delays gastric | Limited data in |
| (US) | 0.6, 1.2 or 1.8 mg | medullary thyroid carcinoma. | emptying. May | patients with |
| liraglutide | once daily | Postmarketing reports of pancreatitis, renal | impact absorption of | renal or hepatic |
| (N,N-2211) | | impairment and serious hypersensitivity | concomitantly | impairment. |
| Novo Nordisk | | reactions (e.g., anaphylactic reactions and | administered oral | Do not use in |
| | | angioedema). | medications. | patients with a |
| | | Increased risk of hypoglycemia when used in | | personal or |
| | | combination insulin or an insulin | | family history of |
| | | secretagogue. | | medullary |
| | | The most common adverse reactions, | | thyroid |
| | | reported in ≧5% of patients treated with | | carcinoma or in |
| | | Victoza and more commonly than in patients | | patients with |
| | | treated with placebo, are: headache, | | Multiple |
| | | nausea, diarrhea and anti-liraglutide | | Endocrine |
| | | antibody formation | | Neoplasia |
| | | Immunogenicity-related events, including | | syndrome type |
| | | urticaria, were more common among | | 2. |
| | | Victoza-treated patients (0.8%) than among | | Do not use if |
| | | comparator-treated patients (0.4%) in | | history of serious |
| | | clinical trials | | hypersensitivity |
| | | Label dated 13 Jun. 2013, US FDA website. | | to Victoza or any |
| | | | | product |
| | | | | components |
| Dulaglutide | Subcutaneous | In clinical trials: | | |
| (LY2189265) | 1.5 mg once | Five Ph III clinical trials (AWARD 1-5) will | | |
| Eli Lilly and Co | weekly | support registration filings expected in | | |
| | | 2013/2014 | | |
| | | AWARD (Assessment of Weekly | | |
| | | AdministRation of LY2189265 in Diabetes) | | |
| | | Gastrointestinal adverse events reported | | |
| CJC-1131 | Subcutaneous | No longer in clinical development | | |
| (DAC ™:GLP-1) | Phll | CJC-1131 is a human GLP-1 analogue with a | | |
| Conjuchem | monotherapy | reactive chemical linker at the Cys 34 | | |
| | trial: | residue which permits covalent coupling to | | |
| | once a day | albumin following administration in vivo. | | |
| | (OD), three | Nausea and vomiting was dose-limiting with | | |
| | times a week | patients in the once a week cohort only able | | |
| | (EOD), | to attain, on average, approximately 35% of | | |
| | twice a week | the target dosing levels. Nevertheless, all | | |
| | (TW), once a | patients achieved significant improvements | | |
| | week (OW) | in glycemic control, with the best results | | |
| | | observed in the once daily dosing cohort. | | |
| LY-307161 | Subcutaneous | LY307161 is a DPP IV-resistant GLP-1 | | |
| (GLP-1 (7-37)) | once-daily | analogue | | |
| Eli Lilly and Co | | | | |
| CJC-1134 | Subcutaneous | | | |
| (exendin-4- | Up to 5 mg | | | |
| albumin | Once weekly | | | |
| conjugate) | Half-life: | | | |
| Conjuchem | 8 days | | | |
| albiglutide | Subcutaneous | Albiglutide is a recombinant-GLP-1 protein | | |
| (GSK716155; | Once weekly | The Phase III clinical development | | |
| albugon) | | programme for albiglutide comprised eight | | |
| Glaxo | | individual studies (Harmony 1 to Harmony 8) | | |
| SmithKline | | involving over 5,000 patients. The | | |
| | | programme investigated the efficacy, | | |
| | | tolerability and safety, including | | |
| | | cardiovascular safety, of albiglutide as | | |
| | | mono- and add-on therapy, in patients with | | |
| | | type 2 diabetes. All eight studies have | | |
| | | completed. | | |
| | | Albiglutide has been submitted for U.S. and | | |
| | | European regulatory approval (FDA decision | | |
| | | expected by 15 Apr. 2014) | | |
| | | The most commonly reported adverse | | |
| | | effects in the studies were gastrointestinal, | | |
| | | mainly nausea and diarrhea, and injection | | |
| | | site reactions | | |
| GLP-1 (7-36) | continuous | GLP-1 (7-36) amide is not very useful for | | |
| amide | subcutaneous | treatment of T2D, since it must be | | |
| | infusion | administered by continuous subcutaneous | | |
| | | infusion. | | |
| GLP1 | Half-life: | The half-life of native GLP-1 is only about 5 | | |
| | 5 minutes | minutes, as it is simultaneously degraded by | | |
| | | serum enzymes and cleared through renal | | |
| | | excretion | | |
| taspoglutide | Subcutaneous | In Phase III clinical trials, there was a high | | |
| Hoffman-La | 10 mg once | patient drop-out rate. The main problems | | |
| Roche | weekly | were gastrointestinal (nausea/vomiting), but | | |
| | | systemic allergic and injection-site reactions | | |
| | | were also more common with taspoglutide | | |
| | | (compared to exenatide); 49% of patients | | |
| | | showed antibodies against the drug. | | |

The combination of a VEGF-B antagonist with a DPP-4 inhibitor or a GLP-1R agonist can reduce the frequency of dosing of DPP-4 inhibitor or GLP-1R agonist to for example, once daily, six times per week, five times per week, four times per week, three times per week, twice per week, once per week, once per two weeks, once per three weeks, once per month or even less frequently.

The combination of a VEGF-B antagonist with a DPP-4 inhibitor or a GLP-1R agonist can permit treatment of additional patient populations that are currently excluded from treatment with either drug used alone (e.g. Byetta® (exenatide) is not recommended for use in patients with severe gastrointestinal disease (e.g. gastroparesis)). Lowering the dose of DPP-4 inhibitor or GLP-1R agonist can permit their use in children under 18 years of age and/or in pregnant women or nursing mothers.

The combinations, including the preferred combinations, are also considered useful in treating metabolic syndrome or dyslipidemias.

Metabolic syndrome (also referred to as syndrome X) is a cluster of risk factors that is responsible for increased cardiovascular morbidity and mortality. Metabolic syndrome is typically characterized by a group of metabolic risk factors that include 1) central obesity; 2) atherogenic dyslipidemia (blood fat disorders comprising mainly high triglycerides (“TG”) and low HDL-cholesterol (interchangeably referred to herein as “HDL”) that foster plaque buildups in artery walls); 3) raised blood pressure; 4) insulin resistance or glucose intolerance (the body can't properly use insulin or blood sugar); 5) prothrombotic state (e.g., high fibrinogen or plasminogen activator inhibitor in the blood); and 6) a proinflammatory state (e.g., elevated high-sensitivity C-reactive protein in the blood). The National Cholesterol Education Program (NCEP) Adult Treatment Panel (ATP) III guidelines define metabolic syndrome by the following five clinical parameters: a) a waist circumference greater than 102 cm for men, and greater than 88 cm for women; b) a triglyceride level greater than 150 mg/dl; c) an HDL-cholesterol less than 40 mg/dl for men, and less than 50 mg/dl for women; d) a blood pressure greater than or equal to 130/85 mmHG; and e) a fasting glucose greater than 110 mg/dl.

As used herein, dyslipidemia is an abnormal serum, plasma, or blood lipid profile in a subject. An abnormal lipid profile may be characterized by total cholesterol, low density lipoprotein (LDL)-cholesterol, triglyceride, apolipoprotein (apo)-B or Lp(a) levels above the 90.sup.th percentile for the general population or high density lipoprotein (HDL)-cholesterol or apo A-1 levels below the 10^{th} percentile for the general population. Dyslipidemia can include hypercholesterolemia and/or hypertriglyceridemia. Hypercholesterolemic human subjects and hypertriglyceridemic human subjects are associated with increased incidence of cardiovascular disorders. A hypercholesterolemic subject has an LDL cholesterol level of >160 mg/dL, or >130 mg/dL and at least two risk factors selected from the group consisting of male gender, family history of premature coronary heart disease, cigarette smoking, hypertension, low HDL (<35 mg/dL), diabetes mellitus, hyperinsulinemia, abdominal obesity, high lipoprotein, and personal history of a cardiovascular event. A hypertriglyceridemic human subject has a triglyceride (TG) level of >200 mg/dL.

The present disclosure is further illustrated by the following non-limiting examples.

### Examples

### Methods Summary

Animals.

The C57BL/6-Vegfb^{-/-} mice have previously been described (Ref 19). C57BKS/Lepr^{db} (db/db) mice were purchased from Jackson Laboratory and bred with Vegfb^{-/-} mice. Db/db//Vegfb^{-/-} mice were bred by mating heterozygous db/+//Vegfb^{+/-} mice with each other. Wt C57BL/6 mice from the Vegfb^{-/-} colony were used as mouse lean controls. Age and sex-matched mice of both sexes were used in all studies unless otherwise stated. Endpoint analyses for lean, db/db, db/db//Vegfb^{+/-} and db/db//Vegfb^{-/-} was at XX weeks of age unless indicated otherwise. For mouse HFD-studies, male wt and Vegfb^{-/-} mice where fed 60% HFD (Research Diets, USA) for 16 weeks from 5 weeks of age. For rat HFD-studies, male Wistar rats were fed a 60% HFD (Speciality Feeds, Western Australia) for 8 weeks from 8 weeks of age. All animals had independent of diet ad libitum access to chow and water, and were housed in standard cages in an environment with 12 hrs light/12 hrs dark cycles. All animal work using mice was conducted in accordance to the Swedish Animal Welfare Board at Karolinska Institutet, Stockholm, Sweden, whereas the animal work using rats was conducted according to relevant national and international guidelines and approved by the Austin Health Animal Ethics Committee, Melbourne, Australia (AEC A2009/03967).

Glucose and Weight Measurements.

Weight and postprandial blood glucose levels of mice were monitored bi-weekly after removal of food for 2 hrs. Glucose measurements were performed on blood drawn from the tail vein using a Bayer Contour Glucose meter. For the measurements of metabolites in fasted plasma, food was removed overnight for a maximum of 16 hrs, glucose was measured under anaesthesia using isofluorane, and the mice were sacrificed afterwards. For analysis of glucosuria, mice were starved for 1 hrs, urine was then collected and the glucose concentration was directly measured using reagent strips according to the manufactures (Uristix, Siemens).

Measurement of Food Intake.

The food intake of lean, HFD-fed and db/db mice as well as HFD-fed rats was measured for 5-7 consecutive weeks. All animals and the amount of food in the cage were weighed once a week. The intake of food presented as g/day/animal was calculated by dividing grams of food eaten per day per amount of animals in the cage. The average of all weeks was presented in the figures.

Metabolic Analyses of Mouse Plasma.

Mice in a postprandial state were anaesthetised with isofluorane, and total blood was withdrawn from the cardiac cavity of mice under deep anaesthesia. The blood was centrifuged for 14000 rpm at 4° C. for 10 minutes, where after plasma was separated and frozen in aliquots in −80° C. Commercially available kits were used for enzymatic determination of NEFAs (Wako Chemicals, Neuss, Germany), beta-hydroxybutyrate (Stanbio Laboratories, Boerne, Tex., USA), triglycerides (Sigma-Aldrich, Germany), and HDL-c and LDL-c (BioVision, Mountain View, Calif., USA).

Phenotyping of Mice.

Analysis of muscular and cardiac mRNA expression, ORO staining and [¹⁸F]FDG uptake by PET were performed as previously described (Hagberg, C. E., et al. Nature 464, 917-921 (2010)).

2H10 Treatment of Db/Db Mice.

7-week or 16-week old db/db mice were injected i.p. twice weekly for 10 weeks with 400 μg of either 2H10 or isotype-matched control antibody. After 9 weeks of treatment, glucose tolerance was evaluated by IPGTT (see below). At the end of the treatment period, the mice were anaesthetised by isofluorane, blood glucose was measured and total blood was removed by cardiac puncture. Organs were harvested and flash frozen for later analyses.

2H10 Treatment of HFD-Fed Wistar Rats.

To minimise the potential immunogenicity of the mouse 2H10 antibody it was reformatted as a chimeric mouse/rat 2H10 IgG antibody. The genes for both the light and heavy chain variable region of the murine anti-VEGFB antibody 2H10²⁸ were synthesized (Geneart) and cloned into separate pcDNA3.1(+) expression vectors which had been modified to contain rat IgG2a and rat kappa constant regions respectively. All animals were treated with either anti-VEGF-B chimaeric mouse/rat 2H10 antibody or an isotype control using a 20 mg/kg dose injected i.p. twice weekly for the 8 week period of the study.

IPGTTs and IPITTs.

IPGTT (intraperitoneal glucose tolerance test) or IPITT (intraperitoneal insulin tolerance test) on mice were performed in the morning on non-fasted mice that had the food removed 1 hr prior to the experiment and injected i.p. (IPGTT 1 mg glucose/g body weight (BW) and IPITT 1 mU insulin/g BW). Pooled data from ≧4 individual experiments are shown.

IPGTTs on the treated rats were performed as previously described but modified (Ref 31, 32, 33). Briefly, rats were fasted for 6 hrs and anaesthetised with an i.p. injection of sodium pentabarbitone (60 mg/kg). Surgery to insert a carotid catheter was performed as below. A basal blood sample was taken prior to an i.p. injection of glucose (2 &kg body weight). Subsequent blood samples were taken at 15, 30, 45, 60 and 120 minutes, centrifuged and stored at −20° C. until analyses of plasma glucose and insulin levels. Red blood cells were returned to the rats with equal volumes of heparinised saline between each sample time to prevent anaemic shock.

Histological Analyses of Pancreas.

Pancreas from antibody-treated db/db mice were dissected and post-fixated in 4% PFA for 48 hrs, subsequently processed for paraffin imbedding using standard procedures, and thereafter immunostained for insulin, glucagon and/or cleaved caspase 3. Briefly, antigen retrieval was performed on 12 μm sections using Antigen retrieval solution PH6 (Dako #S2367) and heated 98C.° for 10 minutes. Sections were incubated at +4° C. for 12 hrs with primary antibodies rat anti-insulin (R&D systems #MAB1417), rabbit anti-glucagon (Millipore #AB932) or rabbit anti-Cleaved caspase-3 (Cell Signalling ASP175). Appropriate fluorescently labelled secondary antibodies (Invitrogen, Alexa fluor) were applied and sections were further incubated for 1 hr at RT after which they were prepared for microscopy.

All pancreatic islets stained for insulin and glucagon within each section were photographed with an Axio Vision microscope (Carl Zeiss) at 20× magnification. The islets were quantified using Axio Vision Run wizard program for i) total islet insulin staining (pixels) ii) total islet glucagon staining (pixels) iii) glucagon staining within the inside core of the pancreatic islet (pixels). Finally, the area of each of the pancreatic islets was measured.

Triglyceride Content in Isolated Pancreatic Islets.

Pancreatic islets were analysed as described (Ref 34). TGs were extracted with a previously published but modified protocol (Ref 35). Briefly, TGs were extracted in a high salt buffer (2 M NaCl, 2 mM EDTA 50 mM sodium phosphate) where after a solution of chloroform methanol (2:1) was added and the sample sonicated for 3×30 sec. After centrifugation (4000 rpm for 15 min) the chloroform phase was collected and dried in nitrogen airflow and reconstituted in 30 μl of isopropanol. TG content in the pancreatic islets was measured by a TG kit (Infinity Triglyceride Reagent, Thermo Scientific) using a standard (Sigma) curve with isopropanol as a reference.

Surgery of Rats for Basal Turnover and Hyperinsulinaemic/Euglycaemic Clamps.

Following an overnight fast, rats were anaesthetised with an i.p. injection of sodium pentobarbitone (60 mg/kg), and heparinised saline (25 U/ml) filled polyethylene catheters (Chrichley Electrical, NSW, Australia) were inserted into the right jugular vein and left carotid artery. The venous catheter was used for infusion and the carotid catheter for blood sampling. A tracheostomy was performed to prevent upper airway obstruction. Body temperature was maintained at 37° C. and monitored throughout with a rectal temperature probe. Anaesthesia was adequately maintained throughout the procedures.

Hyperinsulinaemic/Euglycaemic Clamps and Glucose Uptake into Peripheral Tissues.

All rats were infused with an initial 2 minute priming dose of radio-labeled glucose tracer [6-³H]-glucose at a rate of 100 μBq.min⁻¹ in 0.9% saline followed by a constant infusion of tracer at a rate of 5.5μBq.min⁻¹ in 0.9% saline for the duration of the experiment (155 minutes in total) as previously described (Mangiafico, S. P., et al. J Endocrinol 210, 335-347 (2011), Lamont, B. J., et al. Diabetologia 46, 1338-1347 (2003), Visinoni, S., et al. Am J Physiol Endocrinol Metab 295, E1132-1141 (2008)). Insulin (Actrapid®, Novo Nordisk, Bagsvaerd, Denmark) was infused during the clamp at a dose of 4 mU.kg⁻¹. Blood glucose was maintained at euglycaemia with a 5% glucose solution for the Control HFD group and a 10% glucose solution for the Control Chow and 2H10 HFD groups. Plasma samples were taken at 90, 100 and 110 minutes. To determine glucose uptake into muscle and heart tissues, the labeled 2-[1-¹⁴C]-deoxyglucose technique was used as previously described (Mangiafico, S. P., et al. J Endocrinol 210, 335-347 (2011), Lamont, B. J., et al. Diabetologia 46, 1338-1347 (2003), Kraegen, E. W., et al. Diabetes 40, 1397-1403 (1991), Nolan, C. J. & Proietto, J. et al. Diabetologia 37, 976-984 (1994)).

Determination of Whole Body Glucose Turnover in Rats.

To determine whole body glucose turnover, rat plasma samples collected at 90, 100 and 110 minutes during the hyperinsulinaemic/euglycaemic clamp were treated, processed and measured for the level of [6-³H]-glucose radioactivity as previously described (Mangiafico, S. P., et al. J Endocrinol 210, 335-347 (2011), Lamont, B. J., et al. Diabetologia 46, 1338-1347 (2003), Nolan, C. J. & Proietto, J. et al. Diabetologia 37, 976-984 (1994)). Briefly a 25 μl aliquot of plasma was treated with equal volumes of 0.3 M barium hydroxide and 0.3 M zinc sulphate, centrifuged and 50 t1 of supernatant passed through an anion exchange column (1.5 ml of Dowex 2X8-400 anion exchanger) in order to remove labelled lactate and pyruvate. Glucose was eluted from the column with 4 ml of milliQ water and 10 ml of scintillant (Ultima Gold, PerkinElmer, USA) added and radioactivity determined using a Packard 1900CA TriCarb liquid scintillation analyser (Packard, Meriden, Conn., USA).

Statistics.

In all figures data is presented as mean±SEM from pooled data of 2-3 independent experiments. P-values were calculated with one-way ANOVA and two-tailed independent Student's t-tests, and p<0.05 was considered significant.

1. A method for treating dyslipidemia or a diabetic condition comprising administering a VEGF-B antagonist in combination with an insulin secretagogue selected from a DPP-4 inhibitor and a GLP-1R agonist.

2. The method of claim 1, wherein the VEGF-B antagonist is selected from the anti-VEGF-B antibodies 1C6, 2F5, 2H10 and 4E12, and humanized, deimmunized or chimeric forms thereof.

3. The method of claim 1, wherein the VEGF-B antagonist is a humanized 2H10 antibody comprising SEQ ID NO: 1 and/or SEQ ID NO: 2.

4. The method of claim 1, wherein the VEGF-B antagonist is an antibody that has a binding affinity for human VEGF-B that is substantially equivalent to or stronger than the binding affinity of mAb 2H10 for human VEGF-B.

5. The method of claim 1, wherein the insulin secretagogue is a DPP-4 inhibitor.

6. The method of claim 5, wherein the DPP-4 inhibitor is selected from vildagliptin, sitagliptin, saxagliptin, linagliptin, dutogliptin, gemigliptin, alogliptin, and berberine.

7. The method of claim 1, wherein the insulin secretagogue is a GLP-1R agonist.

8. The method of claim 7, wherein the GLP-1R agonist is selected from exenatide, liraglutide, CJC-1131, LY-307161, dulaglutide, CJC-1134, albiglutide, and taspoglutide.

9. A method for treating dyslipidemia or a diabetic condition comprising administering a VEGF-B antagonist to a subject in need thereof.

10. The method of claim 9, wherein the VEGF-B antagonist is selected from the anti-VEGF-B antibodies 1C6, 2F5, 2H10 and 4E12, and humanized, deimmunized or chimeric forms thereof.

11. The method of claim 9, wherein the VEGF-B antagonist is a humanized 2H10 antibody comprising SEQ ID NO: 1 and/or SEQ ID NO: 2.

12. The method of claim 9, wherein the VEGF-B antagonist is an antibody that has a binding affinity for human VEGF-B that is substantially equivalent to or stronger than the binding affinity of mAb 2H10 for human VEGF-B.

13. A composition for treating diabetic conditions comprising a VEGF-B antagonist in combination with an insulin secretagogue selected from a DPP-4 inhibitor and a GLP-1R agonist.

14. The composition of claim 13, wherein the the VEGF-B antagonist is selected from the anti-VEGF-B antibodies 1C6, 2F5, 2H10 and 4E12, and humanized, deimmunized or chimeric forms thereof.

15. The composition of claim 13, wherein the VEGF-B antagonist is a humanized 2H10 antibody comprising SEQ ID NO: 1 and/or SEQ ID NO: 2.

16. The composition of claim 13, wherein the VEGF-B antagonist is an antibody that has a binding affinity for human VEGF-B that is substantially equivalent to or stronger than the binding affinity of mAb 2H10 for human VEGF-B.

17. The composition of claim 13, wherein the insulin secretagogue is a DPP-4 inhibitor.

18. The composition of claim 17, wherein the DPP-4 inhibitor is selected from vildagliptin, sitagliptin, saxagliptin, linagliptin, dutogliptin, gemigliptin, alogliptin, and berberine.

19. The composition of claim 13 wherein the insulin secretagogue is a GLP-1R agonist.

20. The composition of claim 19, wherein the GLP-1R agonist is selected from exenatide, liraglutide, CJC-1131, LY-307161, dulaglutide, CJC-1134, albiglutide, and taspoglutide.
